(19) United States
(12) Patent Application Publication
(10) Pub. No.: US 2012/0265088 A1
(43) Pub. Date: Oct. 18, 2012

(54) SYSTEM AND METHOD FOR MONITORING OPERATION OF A CARDIAC MEDICAL DEVICE

(75) Inventor: Jeffery D. Snell, Chatsworth, CA (US)

(73) Assignee: PACESETTER, INC., Sylmar, CA (US)

(21) Appl. No.: 13/086,202

(22) Filed: Apr. 13, 2011

(51) Int. Cl. A61B 5/0402

(52) U.S. Cl. 600/523

(57) ABSTRACT

A cardiac monitoring system includes a communication subsystem, a comparison module, and a display module. The communication subsystem receives literal data from a cardiac medical device. The literal data includes cardiac signals and marker data. The cardiac signals represent electrical activity of a heart that is sensed by the medical device. The marker data represents one or more algorithms running on the medical device. The comparison module compares the cardiac signals and marker data to one or more heuristic rules to derive heuristic information about the cardiac signals and the marker data. The heuristic information represents a relationship among the cardiac signals and the marker data. The display module directs a display device to visually present the cardiac signals and a visual indicator representative of the heuristic information. The heuristic information can assist an operator, such as a physician, in changing one or more algorithms running on the medical device.

### FIELD OF THE INVENTION

One or more embodiments described herein generally relate to monitoring systems for implantable and external cardiac medical devices.

### BACKGROUND OF THE INVENTION

Cardiac medical devices monitor, among other things, electrical activity of a heart. Some medical devices may be implanted in patients to also deliver appropriate electrical therapy, such as stimulus pulses to the heart, as required. Implantable medical devices (IMDs) include, for example, pacemakers, cardioverters, defibrillators, implantable cardioverter defibrillators (ICD), and the like. External cardiac medical devices include, for example, programmers, electrocardiogram devices (ECG or EKG), and the like.

These medical devices sense cardiac signals that represent the electrical activity of the heart. The cardiac signals may be displayed on a display device, such as a computer monitor or printed on paper for review by an operator, such as a physician. The cardiac signals are displayed for the physician to review and analyze. In order to analyze the cardiac signals, the physician may be limited to using devices such as physical or electronically generated calipers that measure temporal relationships or spacing between waveform segments and other events represented by the cardiac signals.

Known techniques for analyzing the cardiac signals may be unable to identify certain sequences or patterns in the cardiac signals. For example, some sequences of cardiac events are associated with poor hemodynamic performance. The sequences of cardiac events may be difficult for a physician to quickly identify when the cardiac signals are visually presented to the physician. Moreover, some sequences of cardiac events have relatively complex causes, such as algorithms running on the medical devices that may interfere with each other. Identification of these causes by visually examining the cardiac signals may be difficult for a physician to accomplish.

### BRIEF SUMMARY OF THE INVENTION

In one embodiment, a cardiac monitoring system is provided. The system includes a communication subsystem, a comparison module, and a display module. The communication subsystem receives literal data from a cardiac medical device. The literal data includes cardiac signals and marker data. The cardiac signals represent electrical activity of a heart that is sensed by the medical device. The marker data represents one or more algorithms running on the medical device. The comparison module compares the cardiac signals and the marker data to one or more heuristic rules to derive heuristic information about the cardiac signals and the marker data. The heuristic information represents a relationship among the cardiac signals and the marker data. The display module directs a display device to visually present the cardiac signals and a visual indicator representative of the heuristic information. The heuristic information assists an operator, such as a physician, in changing one or more algorithms running on the medical device.

In another embodiment, a method for monitoring a cardiac medical device is provided. The method includes receiving literal data from the medical device. The literal data includes cardiac signals and marker data. The cardiac signals are representative of electrical activity of a heart that is sensed by the medical device. The marker data represents one or more algorithms running on the medical device. The method also includes comparing the cardiac signals and the marker data to one or more heuristic rules to derive heuristic information about the literal data. The heuristic information represents a relationship between the cardiac signals and the marker data. The method further includes visually presenting the cardiac signals and a visual indicator representative of the heuristic information. The heuristic information assists an operator, such as a physician, in changing one or more of the algorithms running on the medical device.

In another embodiment, a computer readable storage medium for a monitoring system of a cardiac medical device is provided. The monitoring system has a processor and a display device. The storage medium includes one or more sets of instructions that direct the processor to receive literal data from a cardiac medical device. The literal data includes cardiac signals and marker data. The cardiac signals are representative of electrical activity of a heart that is sensed by the medical device. The marker data represents one or more algorithms running on the medical device. The instructions also direct the processor to compare the cardiac signals and the marker data to one or more heuristic rules to derive heuristic information about the cardiac signals and the marker data. The instructions direct the display device to visually present the cardiac signals and a visual indicator representative of the heuristic information. The heuristic information represents a relationship between the cardiac signals and the marker data to assist an operator in changing one or more algorithms running on the medical device.

### BRIEF DESCRIPTION OF THE DRAWINGS

The drawings illustrate generally, by way of example, but not by way of limitation, various embodiments discussed in the present document.

FIG. 1 illustrates one embodiment of a cardiac monitoring system.

FIG. 2 illustrates several cardiac signals and several markers received from a medical device in accordance with one embodiment

FIG. 3 is an illustration of one embodiment of a display presented by a display device of the monitoring system shown in FIG. 1.

FIG. 4 is an illustration of another embodiment of a display presented by the display device of the monitoring system shown in FIG. 1.

FIG. 5 is an illustration of another embodiment of a display presented by the display device of the monitoring system shown in FIG. 1.

FIG. 6 illustrates a functional block diagram of one embodiment of the monitoring system shown in FIG. 1.

FIG. 7 illustrates a distributed processing system in accordance with one embodiment.

FIG. 8 is a flowchart of one embodiment of a method for monitoring a medical device.

### DETAILED DESCRIPTION OF THE INVENTION

In the following detailed description, reference is made to the accompanying drawings which form a part hereof, and in which are shown by way of illustration specific embodiments in which the described subject matter may be practiced. These embodiments, which are also referred to herein as “examples,” are described in sufficient detail to enable one of ordinary skill in the art to practice the claimed subject matter. It is to be understood that the embodiments may be combined or that other embodiments may be utilized, and that structural, logical, and electrical variations may be made without departing from the scope of the disclosed subject matter. For example, embodiments may be used with a pacemaker, a cardioverter, a defibrillator, ECG/EKG, and the like. The following detailed description is, therefore, not to be taken in a limiting sense, and the scope of the claimed subject matter is defined by the appended claims and their equivalents. In this document, the terms “a” or “an” are used, as is common in patent documents, to include one or more than one. In this document, the term “or” is used to refer to a nonexclusive or, unless otherwise indicated.

In accordance with certain embodiments, a cardiac medical device senses cardiac signals of a heart and generates marker data representative of cardiac events, settings of the medical device, algorithms used by the medical device, stimulus pulses delivered by the medical device, etc. The cardiac signals and/or marker data may be referred to herein as “literal data” and may be data that represents the cardiac events, settings, algorithms, stimulus pulses, and the like. For example, the literal data may not represent or include patterns of the events or stimulus pulses, comparisons between subsets of the literal data, and the like.

The monitoring system receives the literal data from the medical device and analyzes the literal data to derive heuristic information from the literal data. The heuristic information can include conclusions or diagnoses that are determined based at least in part on the literal data. In one embodiment, the conclusions or diagnoses are based on a knowledge base or knowledge domain that includes rules or criteria associated with the heuristic information and to which the literal data is compared. The heuristic information may be based on one or more rules, medical standards, or industry standards, and/or may be modified on a physician-by-physician basis, a patient-by-patient basis, a hospital-by-hospital basis, etc. For example, the heuristic information can be customized based on the physician, patient, hospital, and the like. If literal data satisfies or meets the requirements of a rule or criteria, then the heuristic information associated with the rule or criteria is presented to a user of the monitoring system, such as a physician. By way of example, the heuristic information can include: (i) patterns in the cardiac signals and/or marker data that may otherwise be difficult for a physician to quickly identify, (ii) diagnoses of potential cardiac disease and other causes of relatively poor hemodynamic performance or cardiac output of a heart, (iii) identification of pacing or sensing algorithms used by the medical device that are interfering with each other or with the hemodynamic performance of the heart, (iv) recommended changes to settings or algorithms of the medical device.

The heuristic information can be visually presented to a physician as an additional tool to identify cardiac disease or poor hemodynamic performance. Using the heuristic information, the monitoring system may identify patterns of cardiac events that are missed or incorrectly identified by the medical device. In one embodiment, the monitoring system visually presents the cardiac signals obtained by the medical device along with visual indicia representative of the heuristic information. The visual indicial may overlie the cardiac signals and provide visual cues to the operator that enable faster and/or easier identification of cardiac events or patterns of cardiac events.

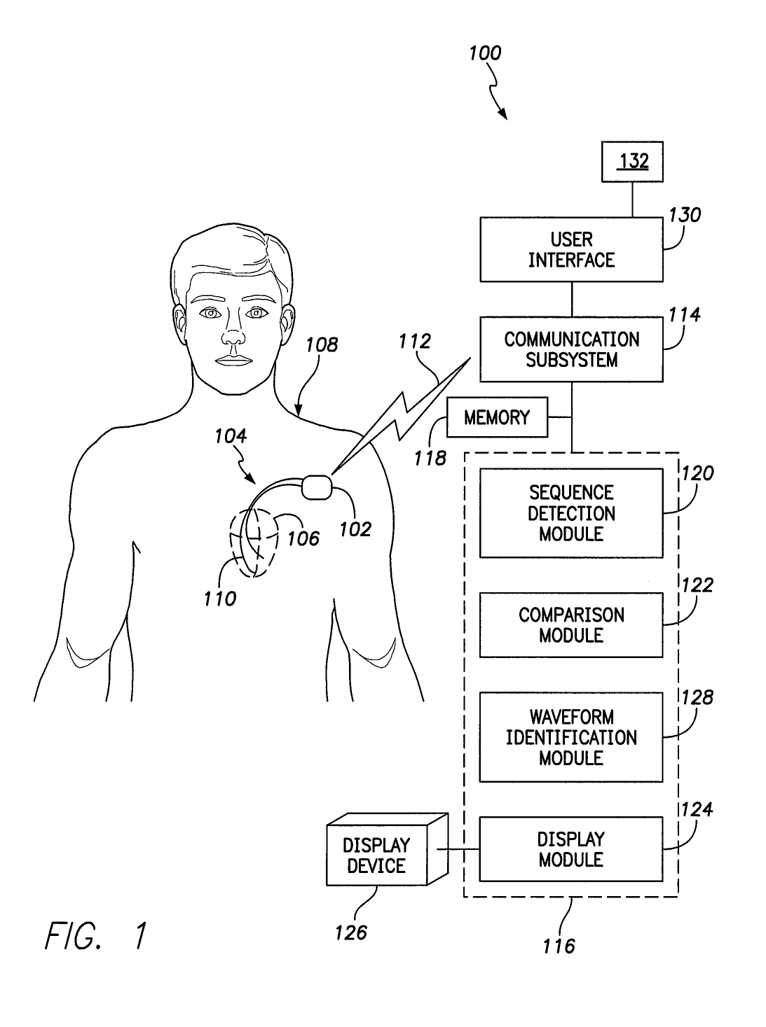

FIG. 1 illustrates one embodiment of a cardiac monitoring system 100. The system 100 communicates with an implantable medical device (IMD) 102 that senses cardiac signals of a heart 106 of a patient 108. The cardiac signals represent electrical activity of the heart 106. In one embodiment, the IMD 102 delivers stimulus pulses to the heart 106 through one or more leads 110 implanted in the heart 106. By way of example only, the IMD 102 may be a cardiac pacemaker, an ICD, a defibrillator, an ICD coupled with a pacemaker, a CRT pacemaker or a cardiac resynchronization therapy defibrillator (CRT-D). In another embodiment, the IMD 102 may not deliver stimulus pulses to the heart 108 and/or may be external to the patient 108. For example, the IMD 102 may be an electrocardiography (ECG or EKG) device that uses electrodes positioned on the skin of the patient 108 to monitor cardiac signals. While only a single IMD 102 is shown, alternatively, two or more medical devices 102 may be used.

The IMD 102 senses the cardiac signals of the patient 108 using sensing algorithms. The sensing algorithms can include instructions that direct the IMD 102 to generate cardiac signals based on the electrical activity of the heart 106. The sensing algorithms may be embodied in one or more software applications that provide logic-based directions for the IMD 102 to follow. For example, different sensing algorithms may have different settings, such as sensing thresholds that are compared to the sensed cardiac signals to identify cardiac events. When the cardiac event exceeds a threshold, a cardiac event, such as a waveform segment of interest, is identified by the IMD 102.

Other settings of the sensing algorithms may include time period thresholds. The IMD 102 can measure an amount of time that elapses between cardiac and/or pacing events. For example, the IMD 102 may measure an atrioventricular interval (AVI) that represents the time period between atrial and ventricular events. As another example, the IMD 102 may measure a capture interval that represents the time following delivery of a stimulus pulse until a cardiac event is sensed, such as a ventricular contraction representative of capture of the stimulus pulse.

The IMD 102 can employ one or more pacing algorithms to determine when and/or where to deliver stimulus pulses to the heart 106. For example, the IMD 102 may deliver a stimulus pulse to the heart 106 following a cardiac event (or when a cardiac event of interest is not detected for a predetermined time period). The pacing algorithm may determine when and/or where to deliver the stimulus pulses.

The IMD 102 can generate marker data. The marker data may represent when a cardiac event occurs, such as when a waveform segment (e.g., P-wave, R-wave, QRS complex, T-wave, or other waveform segment) occurs. The marker data may represent actions taken by the medical device (e.g., when a stimulus pulse is delivered) and/or settings of the IMD 102, such as thresholds, blanking periods, sensing algorithms, and/or pacing algorithms that are used by the IMD 102. The marker data may be associated with time stamps such that the marker data can be communicated from the IMD 102 to the monitoring system 100 and the monitoring system is able to determine when the IMD 102 identified cardiac events occurred and/or when stimulus pulses were delivered by the IMD. As used herein, the marker data that represents when cardiac events of the heart 106 occur is referred to as “cardiac markers.” The marker data that represents operations of the medical device, such as the settings, thresholds, pacing algorithms, sensing algorithms, and the like of the IMD 102 is referred to as “operational markers.” The operational markers also may include markers that indicate when and/or where stimulus pulses are delivered to the heart 106. As described above, the cardiac signals sensed by the IMD 102 and the marker data generated by the IMD are referred to as literal data.

The literal data is communicated from the IMD 102 to the monitoring system 100 as transmitted data 112. In one embodiment, the IMD 102 wirelessly transmits the transmitted data 112 as telemetry data. Alternatively, the IMD 102 may be wired to the monitoring system 100 and conveys the transmitted data 112 to the monitoring system 100 over one or more conductive busses or wires.

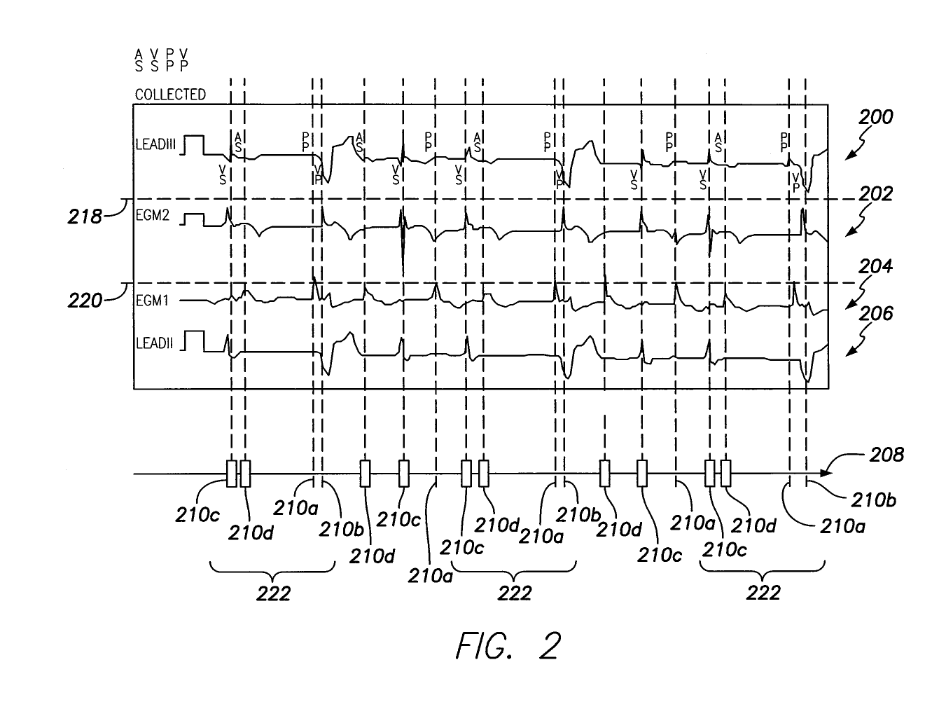

FIG. 2 illustrates several cardiac signals 200, 202, 204, 206 and several markers 210 that are utilized in accordance with one embodiment. The cardiac signals 200, 202, 204, 206 may represent electrical activity of the heart 106 as sensed by a single IMD 102 or a plurality of medical devices. For example, the cardiac signals 200 and 206 may be sensed by different leads 110 of an implantable IMD 102 (such as an ICD) and the cardiac signals 202, 204 may be sensed by different leads of an external IMD 102 (such as an ECG device).

A timeline 208 is shown below the cardiac signals 200, 202, 204, 206. The timeline 208 represents the time period over which the cardiac signals 200, 202, 204, 206 are obtained. Several markers 210 are shown on the timeline 208. The markers 210 include operational markers and cardiac markers. In the illustrated embodiment, the markers 210a are operational markers that represent when a stimulus pulse is applied to an atrium of the heart 106 by the IMD 102. The markers 210a may be referred to as atrial pulse markers. The markers 210b are operational markers that represent when a stimulus pulse is applied to a ventricle of the heart 106 by the IMD 102. The markers 210b may be referred to as ventricular pulse markers.

In the illustrated embodiment, the markers 210c are cardiac markers that represent identification of ventricular events, such as ventricular contraction, by the IMD 102. The cardiac markers 210c may be referred to as ventricular sense cardiac markers. The IMD 102 may detect ventricular contraction and generate a ventricular sense cardiac marker 210c when the cardiac signals 202 exceed a ventricular sense threshold 218. The markers 210d are cardiac markers that represent detection of atrial events, such as atrial contraction, by the IMD 102. The cardiac markers 210d may be referred to as atrial sense cardiac markers. The IMD 102 may detect atrial contraction and generate an atrial sense cardiac marker 210d when the cardiac signals 204 exceed an atrial sense threshold 220 and when no stimulus pulse is applied to the heart 106 or to an atrium within a predetermined time window. Alternatively, the IMD 102 may create an atrial sense cardiac marker 210d when the cardiac signals 204 exceed the atrial sense threshold 220.

The operational markers 210a, 210b and the cardiac markers 210c, 210d are shown on the timeline 208 to represent when the operational markers and the cardiac markers occur relative to each other and to the cardiac signals 200, 202, 204, 206. Returning to the discussion of the monitoring system 100 shown in FIG. 1, the IMD 102 transmits the operational markers 210a, 210b, the cardiac markers 210c, 210d, and the cardiac signals 200, 202, 204, 206 to the monitoring system 100 as the transmitted data 112. The transmitted data 112 represents the literal data conveyed from the IMD 102. For example, the literal data includes the operational markers 210a, 210b, the cardiac markers 210c, 210d, and the cardiac signals 200, 202, 204, 206 in one embodiment.

The monitoring system 100 includes a user interface 130 with an input device 132 (e.g., a keyboard, microphone, stylus, touchscreen, or electronic mouse). The user interface 130 and input device 132 may be used by a physician to control operations of the monitoring system 100. The monitoring system 100 also includes a communication subsystem 114 that receives the transmitted data 112 from the IMD 102. In one embodiment, the communication subsystem 114 includes a telemetry subsystem that wirelessly receives the transmitted data 112 via a telemetry wand or other wireless antenna. Alternatively, the communication subsystem 114 may include a mechanical connector that is conductively coupled with busses or wires to receive the transmitted data 112.

The monitoring system 100 includes a programmable processor 116 that controls analysis and/or presentation of the transmitted data 112 by the monitoring system 100. For example, the processor 116 may control the analysis of the literal data to derive heuristic information about the literal data. The processor 116 may alternatively be referred to as a microprocessor, a controller, a microcontroller, or processing unit. The processor 116 includes a computer processor, or equivalent control circuitry, and may further include RAM or ROM memory, logic and timing circuitry, state machine circuitry, and I/O circuitry. The processor 116 may include one or more modules and/or other processors configured to perform one or more of the operations described herein.

The modules discussed herein may be embodied in sets of instructions that are stored on a computer readable storage medium, such as a memory 118, and that are executed by the processor 116. The memory 118 may be a tangible and non-transitory storage medium, such as one or more computer hard drives, RAM, ROM, flash drives, EEPROM, and the like. The modules discussed below may represent different sets of instructions, such as software applications, that are stored on the memory 118 and executed by the processor 116. Alternatively, the modules may represent different memories that include software applications and/or different processors.

In one embodiment, the cardiac signals and/or the marker data of the IMD 102 can be recorded to the memory 118. The memory 118 may be a removable memory, such as a CD, DVD, flash drive, and the like, that is removable from the system 100. The memory 118 may receive and store cardiac signals and/or marker data from the communication subsystem 114. The memory 118 may then, or at a later time, convey the stored cardiac signals and/or marker data to the processor 116.

In one embodiment, a sequence detection module 120 examines the marker data received from the IMD 102. For example, the sequence detection module 120 may identify a pattern in the cardiac markers and/or operational markers over a period of time. The sequence detection module 120 may identify how many times a cardiac marker, such as the cardiac markers 210c and/or 210d, occurs during the period of time. With respect to the example embodiment shown in FIG. 2, the marker pattern may be represented by the order and/or timing in which the cardiac markers 210c and/or 210d occur. Alternatively, the marker pattern may represent the order and/or timing in which the operational markers 210a and/or 210b occur. In another embodiment, the marker pattern may include a combination of cardiac markers and operational markers.

A comparison module 122 examines the marker pattern identified by the sequence detection module 120 in order to derive heuristic information about the marker data. In one embodiment, the comparison module 122 compares the marker pattern to one or more heuristic rules to determine if the marker pattern satisfies or meets the heuristic rules. For example, the comparison module 122 may compare a plurality of the markers in the marker pattern to several heuristic rules to determine relationships between or among two or more of the markers. Various rules may identify different relationships between the markers. The markers in the marker pattern may satisfy a heuristic rule when the marker pattern matches or corresponds with the marker relationships in the rule. For example, the comparison module 122 can identify temporal relationships between two or more operational markers 210a, 210b and/or cardiac markers 210c, 210d (shown in FIG. 2). The temporal relationship can include a measurement of a time period between a plurality of the markers, a frequency at which one or more markers occurs, a deviation or change in time periods between or among several of the markers, and the like. Other temporal relationships between the markers may be derived by the comparison module 122.

In one embodiment, the user interface 130 may receive input from an operator of the system 100 (e.g., a physician) to modify or change one or more heuristic rules. For example, a physician may change one or more heuristic rules that are used to derive the heuristic information from cardiac signals and/or marker data. The physician may change the heuristic rules based on personal preferences, professional experience, on a patient-by-patient basis, and the like.

With respect to the example of the marker data shown in FIG. 2, the comparison module 122 may measure a ventricular interval as a temporal relationship between the markers related to ventricular events. For example, a ventricular interval may be measured between a ventricular sense cardiac marker 210c or a ventricular pace operational marker 210b and a subsequent ventricular sense cardiac marker 210c or a subsequent ventricular pace marker 210b.

Another temporal relationship that may be identified by the comparison module 122 may be an AVI between two or more of the markers. With respect to the example of the marker data shown in FIG. 2, the comparison module 122 may measure an AVI as a temporal relationship between the cardiac markers that are related to atrial and ventricular events. For example, an AVI may be a measurement of the time elapsed from an atrial sense cardiac marker 210d and a subsequent ventricular sense cardiac marker 210c.

In one embodiment, the comparison module 122 identifies a ventricular-atrial interval (VAI) as a temporal relationship between the marker data. The VAI represents a time period extending from a ventricular event (such as a ventricular contraction or delivery of a stimulus pulse to a ventricle) to an atrial event (such as an atrial contraction or delivery of a stimulus pulse to an atrium). With respect to the example of the marker data shown in FIG. 2, the comparison module 122 may measure a VAI as the time elapsed from a ventricular sense cardiac marker 210c to a subsequent atrial sense cardiac marker 210d.

Another temporal relationship that may be identified by the comparison module 122 is an atrial interval. The atrial interval represents the time period between atrial events. The comparison module 122 may measure an atrial interval as the time elapsed between sensed atrial contractions, or between atrial sense cardiac markers 210d. In another embodiment, the comparison module 122 (shown in FIG. 1) may calculate the time delay between delivery of a stimulus pulse to an atrium or ventricle and contraction in a corresponding atrium or ventricle. This time delay may be measured as another temporal relationship between marker data.

The marker pattern and/or relationships between the marker data may be heuristic information that is presented to a physician in order to aid with the analysis and/or diagnosis of hemodynamic performance of the heart 106. A display module 124 of the monitoring system 100 directs a display device 126, such as a monitor, LCD screen, or other device capable of visually presenting data, to display the heuristic information and/or the cardiac signals. In one embodiment, the display device 126 may be remotely located from the system 100. For example, a physician viewing the display device 126 may be located in a different room, floor, building, city block, ZIP code, city, county, state, country, and the like, from the system 100. The system 100 can interface with the physician via the user interface 130 and the display device 126 and can remotely process the cardiac signals and/or marker data to present the physician with the heuristic information.

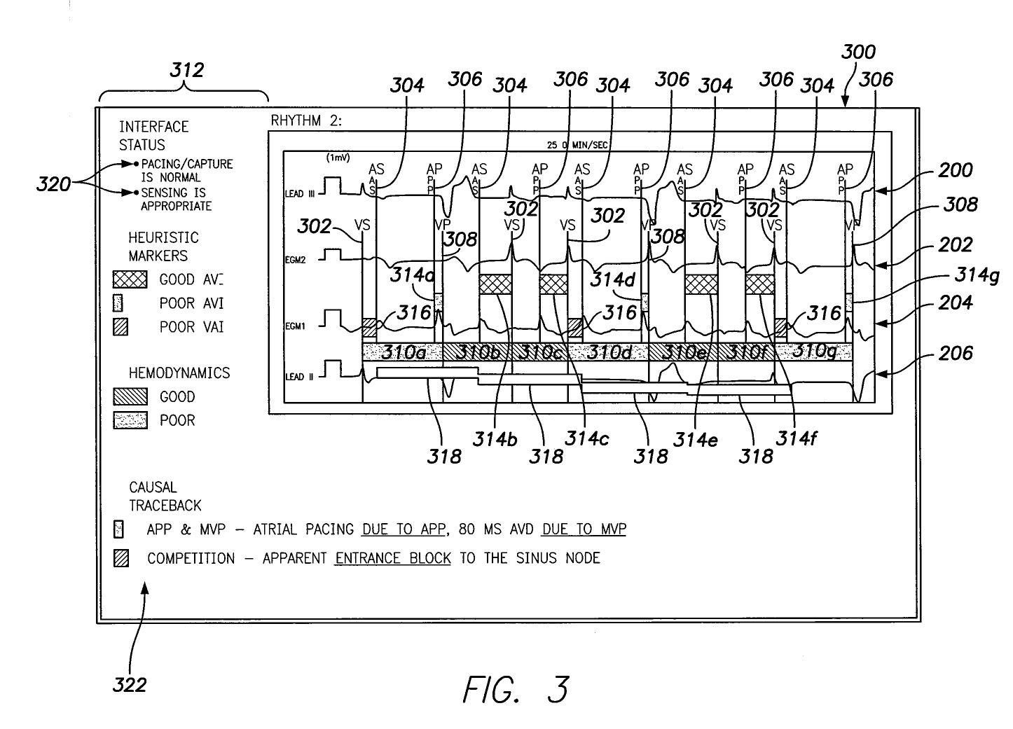

FIG. 3 illustrates a display 300 presented by the display device 126 in accordance with one embodiment. The display 300 shown in FIG. 3 is an example of the heuristic information that may be presented by the display device 126 along with the cardiac signals. In the display 300, the display module 124 causes the display device 126 to visually present the cardiac signals 200, 202, 204, 206 and visual indicia representative of the heuristic information derived by the comparison module 122. In the illustrated embodiment, the visual indicia are shown at least partially overlying, or overlapping, the cardiac signals 200, 202, 204, 206 in order to associate the markers and/or heuristic information represented by the visual indicia with the cardiac signals 200, 202, 204, 206.

The visual indicia shown in FIG. 3 include ventricular sense indicia 302, atrial sense indicia 304, atrial pulse indicia 306, and ventricular pulse indicia 308. While the sense and pace indicia 302, 304, 306, 308 are shown as vertical lines, alternatively a different line or geometric shape or shading may be used. The ventricular sense indicia 302 represent ventricular sense cardiac markers 210c. The atrial sense indicia 304 represent atrial sense cardiac markers 210d. The atrial pulse indicia 306 represent atrial pulse operational markers 210a. The ventricular pulse indicia 308 represent ventricular pulse operational markers 210b. The sense and pace indicia 302, 304, 306, 308 are displayed over the cardiac signals 200, 202, 204, 206 at positions that correspond to the times and/or locations associated with the cardiac markers. The visual presentation of the sense and pace indicia 302, 304, 306, 308 can assist an operator of the monitoring system 100, such as a physician, to easily identify atrial and ventricular events.

The visual indicia also include ventricular interval indicia 310. The ventricular interval indicia 310 correspond to temporal relationships between the marker data. In the illustrated embodiment, the ventricular interval indicia 310 represent the ventricular intervals determined by the comparison module 122. The ventricular interval indicia 310 are presented as elongated areas, or rectangles, that extend between two or more ventricular events, such as between ventricular sense cardiac markers 210c and/or ventricular pace operational markers 210b. Alternatively, the ventricular interval indicia 310 may have a different shape or be visually presented in a different manner. The horizontal or lateral length of the ventricular interval indicia 310 visually represents the length of time between ventricular events, as described above.

In the illustrated embodiment, the ventricular interval indicia 310 include ventricular interval indicia 310a-310g. Each of the ventricular interval indicia 310a, 310d, 310g begins at a ventricular sense indicator 302 and ends at a subsequent ventricular pulse indicator 308. Each of the ventricular interval indicia 310b, 310e begins at a ventricular pulse indicator 308 and ends at a subsequent ventricular sense indicator 302. Each of the ventricular interval indicia 310c, 310f begins at a ventricular sense indicator 302 and ends at a subsequent ventricular sense indicator 302.

The ventricular interval indicia 310 may be presented in different colors, shapes, shadings, and the like, or in different positions relative to each other, such as different vertical positions. For example, the ventricular interval indicia 310a, 310d, and 310g are visually presented in a first color or shading while the ventricular interval indicia 310b, 310c, 310e, and 310f are visually presented in a different second color or shading. The color, shape, shading, position, and the like in which the ventricular interval indicia 310 may be presented indicates additional heuristic information that is determined by the monitoring system 100. For example, the different colors, shades, shapes, positions, and the like, may represent characteristics of the marker data and/or cardiac signals from which the ventricular interval indicia 310 are derived. In the illustrated embodiment, the comparison module 122 of the monitoring system 100 assigns different colors or shades to the different ventricular interval indicators 310 based on the length of time represented by the ventricular interval indicators 310. The comparison module 122 may compare the time periods represented by the ventricular interval indicia 310 to one or more time period thresholds. Based on which thresholds are exceeded by the time periods represented by the ventricular interval indicia 310, the ventricular interval indicia 310 may be assigned to or classified in one of several groups.

The comparison module 122 may classify the derived heuristic information and the display module 124 may change the appearance of one or more visual indicia based on the classification. In the illustrated embodiment, the time periods represented by the ventricular interval indicia 310 are compared to one or more thresholds. The ventricular interval indicia 310 that exceed the threshold (e.g., the ventricular interval indicia 310a, 310d, and 310g) are classified in a first group by the comparison module 122 while the ventricular interval indicia 310 that do not exceed the threshold (e.g., the ventricular interval indicia 310b, 310c, 310e, and 310f) are classified in a different second group. The first group may be referred to as a “poor hemodynamic performance” group, while the second group may be referred to as a “good hemodynamic performance” group. For example, the threshold to which the time periods of the ventricular interval indicia 310 is compared may be a time period that is associated with a sufficiently fast ventricular contraction rate to provide a cardiac output of the heart 106 that exceeds a cardiac output threshold. The ventricular intervals that are shorter than the threshold (e.g., the ventricular interval indicia 310b, 310c, 310e, and 310f) represent sufficiently fast ventricular contraction rates and sufficiently high cardiac output, otherwise referred to as “good hemodynamic performance.” Conversely, the ventricular intervals that are longer than the threshold (e.g., the ventricular interval indicia 310a, 310d, and 310g) represent slower ventricular contraction rates and/or low cardiac output, otherwise referred to as “poor hemodynamic performance.”

The visual presentation of the ventricular interval indicia 310 and/or the different shapes, colors, shading, positions, and the like of the ventricular interval indicia 310 may assist a physician in quickly identifying patterns or problems with the hemodynamic performance of the heart 106. The display device 126 may present a legend 312 that describes the various shape, color, shading, or positional schemes used to represent different heuristic information. In another embodiment, the monitoring system 100 may display only the ventricular interval indicia 310 that are classified in one or more, but less than all, of the groups of ventricular interval indicia 310. For example, the display device 126 may only present those ventricular interval indicia 310 that represent poor hemodynamic performance or health, such as those ventricular intervals that do not meet or exceed one or more medical or industry standards.

The heuristic information may include AVI indicia 314. The AVI indicia 314 correspond to temporal relationships between the marker data. In the illustrated embodiment, the AVI indicia 314 represent the AVIs determined by the comparison module 122 between atrial events and ventricular events, as described above. The AVI indicia 314 are presented as elongated areas, or rectangles, that extend between two or more atrial and ventricular events, such as between atrial pulse operational markers 210a or atrial sense cardiac markers 210d and subsequent ventricular pulse operational markers 210b or ventricular sense cardiac markers 210c. Alternatively, the AVI indicia 314 may have a different shape or be visually presented in a different manner. As shown in FIG. 3, the horizontal or lateral length of the AVI indicia 314 visually represents the length of time between atrial and ventricular events.

In the illustrated embodiment, the AVI indicia 314 include AVI indicia 314a-g. Each of the AVI indicia 314a, 314d, and 314g begins at an atrial pulse indicator 306 and ends at a subsequent ventricular pulse indicator 308. Each of the AVI indicia 314b and 314e begins at an atrial sense indicator 304 and ends at a subsequent ventricular sense indicator 302. Each of the AVI indicia 314c and 314f begins at an atrial pulse indicator 306 and ends at a subsequent ventricular sense indicator 302.

The AVI indicia 314 may be presented in different colors, shapes, shadings, positions, and the like. In the illustrated embodiment, the AVI indicia 314 are presented in different colors and different vertical positions relative to each other based on additional heuristic information related to the AVI indicia 314. For example, the AVI indicia 314a, 314d, and 314g are visually presented in a third color or shading at a first vertical position while the AVI indicia 314b, 314c, 314e, and 314f are visually presented in a different fourth color or shading at a higher second vertical position. The color, shape, shading, position, and the like can represent characteristics of the marker data and/or cardiac signals from which the AVI indicia 314 are derived. In the illustrated embodiment, the display module 124 assigns different vertical positions and colors or shades to the different AVI indicia 314 based on the lengths of time represented by the AVI indicia.

For example, the time periods represented by the AVI indicia 314 can be compared to a time period threshold. The AVI indicia 314 that exceed the threshold (e.g., the AVI indicia 314b, 314c, 314e, and 314f) are classified in a third group by the comparison module 122 while the AVI indicia 314 that do not exceed the threshold (e.g., the AVI indicia 314a, 314d, and 314g) are classified in a fourth group. The first group may be referred to as a “good AVI” group while the second group may be referred to as a “poor AVI” group. For example, the threshold to which the time periods of the AVI indicia 314 is compared may be a time period that represents an AVI associated with good cardiac performance or health, such as those AVIs that meet or exceed one or more medical or industry standards. The AVIs that are shorter than the threshold represent AVIs that are faster than the AVI and are not representative of good cardiac performance or health, such as those AVIs that do not meet medical or industry standards. The AVIs that are longer than the threshold may represent AVIs that are at least as long as the AVI representative of good cardiac performance or health, such as those AVIs that meet medical or industry standards.

The visual presentation of the AVI indicia 314 and/or the different shapes, colors, shading, or positions of the AVI indicia 314 can assist a physician in quickly identifying patterns or problems with the performance of the heart 106. The heuristic information that is associated with the different shapes, colors, shading, positions, and the like of the AVI indicia 314 can be presented in the legend 312, as shown in FIG. 3. In another embodiment, the monitoring system 100 may display only the AVI indicia 314 that are classified in one or more, but less than all, of the groups of AVI indicia 314. For example, the display device 126 may only present those AVI indicia 314 that represent poor cardiac performance or health.

The heuristic information presented on the display device 126 may include VAI indicia 316. The VAI indicia 316 correspond to temporal relationships between the marker data. In the illustrated embodiment, the VAI indicia 316 represent the VAIs determined by the comparison module 122 between ventricular events and atrial events, as described above. The VAI indicia 316 are presented as colored or shaded areas that extend between two or more atrial and ventricular events, such as between ventricular sense cardiac markers 210c and atrial sense cardiac markers 210d. Alternatively, the VAI indicia 316 may have a different shape or be visually presented in a different manner. The horizontal or lateral length of the VAI indicia 316 may visually represent the length of time between ventricular and atrial events. In the illustrated embodiment, each of the VAI indicia 316 begins at a ventricular sense indicator 302 and ends at a subsequent atrial sense indicator 314.

In one embodiment, the comparison module 122 determines which VAI are representative of poor cardiac performance of the heart 106, such as those VAIs that do not meet or exceed medical or industry standards. For example, the comparison module 122 may compare several VAIs that are calculated based on the cardiac markers and/or cardiac signals to one or more time period thresholds. At least one of the time period thresholds may represent a time period between ventricular and atrial events that is indicative of good cardiac performance or sufficient cardiac performance relative to one or more medical or industry standards. The VAIs that exceed the threshold (e.g., the VAIs that are not represented by VAI indicia 316 in FIG. 3) may meet or exceed the medical or industry standards and, as a result, be classified in a fifth group. The VAIs that do not exceed the threshold (e.g., the VAIs represented by the VAI indicia 316 in FIG. 3) may not meet or exceed the medical or industry standards and, as a result, be classified in a sixth group.

The display module 124 can direct the display device 126 to present one or more of the groups of VAI indicia 316. In the illustrated embodiment, only those VAI indicia 316 that are associated with the VAIs that do not exceed the threshold are shown. Alternatively, different VAI indicia 316 may be presented, such as the VAI indicia 316 associated with the VAIs that exceed the threshold. The visual presentation of the VAI indicia 316 and/or the different shapes, colors, shading, or positions of the VAI indicia 316 can assist a physician in quickly identifying patterns or problems with the performance of the heart 106. The heuristic information that is associated with the different shapes, colors, shading, positions, and the like of the VAI indicia 316 may be presented in the legend 312. For example, in the illustrated embodiment, the displayed VAI indicia 316 are associated with a “poor VAI” group, as shown in the legend 312.

Atrial rate indicia 318 can be heuristic information that is presented on the display device 126. The atrial rate indicia 318 represent atrial intervals, or time periods between atrial events, as described above. The atrial rate indicia 318 are presented as colored or shaded areas that extend between two or more atrial events, such as between atrial sense cardiac markers 210d. Alternatively, the atrial rate indicia 318 may have a different shape or be visually presented in a different manner. The horizontal or lateral length of the atrial rate indicia 318 may visually represent the length of time between atrial events, such as between atrial contractions. Longer atrial rate indicia 318 represent longer time periods between atrial contractions while shorter atrial rate indicia 318 represent shorter time periods between atrial contractions. The number of atrial rate indicia 318 that is shown over a time period may be used to visually estimate or calculate the rate of atrial contraction. For example, faster atrial contraction rates are represented by larger numbers of atrial rate indicia 318 while slower atrial contraction rates are represented by smaller numbers of atrial rate indicia 318 over a time period shown on the display device 126.

The visual presentation of the atrial rate indicia 318 and/or the different shapes, colors, shading, or positions of the atrial rate indicia 318 can assist a physician in quickly identifying patterns or problems with the performance of the heart 106. The heuristic information that is associated with the different shapes, colors, shading, positions, and the like of the atrial rate indicia 318 can be presented in the legend 312. In one embodiment, the color, shape, position, shading, and the like of the atrial rate indicia 318 may vary based on a deviation between the atrial contraction rates that are represented by the atrial rate indicia 318. For example, the atrial rate indicia 318 that are associated with atrial intervals that vary or deviate from a statistical measure of other atrial intervals (e.g., a mean or median of atrial intervals calculated over a moving time window) may be presented in a different color, shape, shading, position, and the like on the display device 126.

Returning to the discussion of the monitoring system 100 shown in FIG. 1, the comparison module 122 may derive diagnostic information from the literal data as additional heuristic information. The diagnostic information may include information that assists a physician in identifying events, patterns, and the like in the literal data received from the IMD 102. The diagnostic information may include potential causes for the events, patterns, and the like in the literal data. For example, the comparison module 122 may determine heuristic information that assists a physician in more rapidly diagnosing cardiac disease or events, and/or prescribe or change programming or settings of the IMD 102.

In one embodiment, the comparison module 122 determines diagnostic information based on one or more marker patterns identified by the sequence detection module 120. As described above, the sequence detection module 120 may identify marker patterns in the cardiac markers and/or operational markers generated by the IMD 102. The memory 118 can store heuristic rules, such as predetermined patterns of cardiac markers and/or operational markers that are associated with diagnostic information. For example, a first pattern of cardiac markers may be associated with diagnostic information that includes a recommended adjustment to the settings or thresholds of the IMD 102 while a second pattern of cardiac markers is associated with a different recommended adjustment to the IMD 102. Additional patterns of cardiac markers, operational markers, and combinations of cardiac and operational markers also may be stored in the memory 118 and associated with other diagnostic information. In addition to or in place of recommended changes to the IMD 102, the diagnostic information may include proposed diagnoses for the physician to consider when treating the patient 108.

The comparison module 122 compares the marker pattern identified by the sequence detection module 120 with one or more of the predetermined patterns in the memory 118 to determine one or more degrees of match between the marker pattern and the predetermined patterns. The degree of match is a measurement of the correlation between the patterns. For example, if the marker pattern and a predetermined pattern include the same cardiac markers occurring at the same time or within a predetermined time period of each other, then the marker pattern and the predetermined pattern may have a 100% degree of match. In another example, if two-thirds of the markers in the marker pattern correlate to the markers in the predetermined pattern, then the degree of match may be 67%. Other degrees of match may be calculated.

The comparison module 122 obtains diagnostic information from the memory 118 based on the degrees of match between the marker pattern and the predetermined patterns. For example, the comparison module 122 may select diagnostic information associated with the predetermined pattern having a larger degree of match than one or more other predetermined patterns. Alternatively, the comparison module 122 may select the diagnostic information associated with two or more predetermined patterns based on the degrees of match. In another embodiment, the degree of match between a predetermined pattern and a marker pattern may be increased based on the number of times the predetermined pattern appears in the marker pattern.

Returning to the discussion of the cardiac signals 202, 204 and the marker pattern shown on the timeline 208 in FIG. 2, and with continued reference to the monitoring system 100, the memory 118 may store a first predetermined pattern of cardiac and operational markers that is compared to the marker data shown on the timeline 208. In one example, the predetermined pattern may include a ventricular sense cardiac marker, a subsequent atrial sense cardiac marker that is 90 to 110 milliseconds following the ventricular sense cardiac marker, an atrial pace operational marker that follows the atrial sense cardiac marker by 480 to 520 milliseconds, and a ventricular pace operational marker that follows the atrial pace operational marker by 75 to 85 milliseconds. Additional predetermined patterns may be stored in the memory 118.

The comparison module 122 compares the first predetermined pattern to the marker pattern shown on the timeline 208. The comparison module 122 determines that the first predetermined pattern has a relatively high degree of match with the marker pattern shown on the timeline 208. For example, the pattern of the ventricular sense cardiac marker 210c, followed by the atrial sense cardiac marker 210d, followed by the atrial pulse operational marker 210a, followed by the ventricular pulse operational marker 210b, along with the time periods between the operational markers 210c, 210d, 210a, 210b, may match the first predetermined pattern with a relatively high degree of match. In the illustrated embodiment shown in FIG. 2, several groups 222 of the markers are labeled to identify the marker patterns that match the first predetermined pattern with a relatively high degree of match.

Once the groups 222 of markers are identified by the comparison module 122, the comparison module 122 retrieves the diagnostic information associated with the predetermined marker pattern from the memory 118. The retrieved diagnostic information is conveyed to the display module 124, which can present the diagnostic information on the display 300 (shown in FIG. 3), such as by presenting text representative of the diagnostic information.

In one embodiment, the predetermined patterns are associated with one or more operational markers that represent sensing and/or pacing algorithms of the IMD 102. In addition to determining whether the marker data matches the predetermined pattern, the comparison module 122 also may compare the operational markers received from the IMD 102 to the operational markers of the predetermined pattern. For example, the comparison module 122 determines if one or more of the sensing or pacing algorithms being used by the IMD 102 match one or more of the sensing or pacing algorithms associated with the predetermined pattern. If the operational markers match, then the comparison module 122 may determine that the diagnostic information associated with the predetermined pattern and the operational markers stored in the memory 118 applies to the literal data (e.g., cardiac signals and marker data) received from the IMD 102.

With respect to the embodiment shown in FIGS. 2 and 3, the operational markers received from the IMD 102 may indicate that the IMD 102 was using pacing algorithms entitled “atrial pacing protocol” (APP) and “managed ventricular pacing” (MVP) protocol. The operational markers associated with the first predetermined pattern include markers that represent the APP and MVP protocols. As a result, the comparison module 122 determines that the marker pattern matches the predetermined pattern and the algorithms used by the IMD 102 match the operational markers associated with the predetermined pattern. The comparison module 122 then obtains the diagnostic information associated with the predetermined pattern and presents the diagnostic information on the display 300. In the illustrated embodiment, the diagnostic information is shown as a causal traceback text 322, which notifies the physician that the marker data indicative of the poor AVI heuristic information may be associated with the APP and MVP protocols while the poor VAI heuristic information may be associated with an apparent entrance block to the sinus node of the heart 106. The physician may use this diagnostic information to alter or change one or more settings or algorithms of the IMD 102.

Additional diagnostic information may be presented as other text 320 in the display 300. This additional diagnostic information may be obtained from the memory 118 by identifying which predetermined patterns match the marker data, as described above.

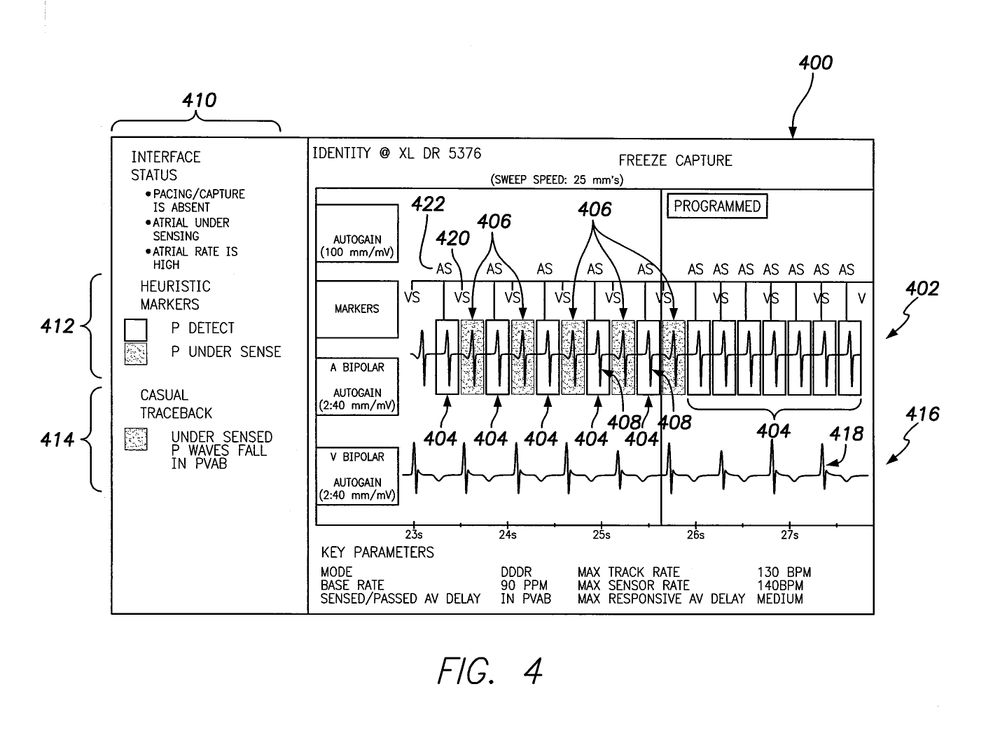

FIG. 4 is an illustration of another embodiment of a display 400 presented by the display device 126. The display 400 is another example of the visual information that may be presented by the display device 126 based on the cardiac signals and marker data from the IMD 102, as well as heuristic information (e.g., relationships between markers) derived by the comparison module 122.

The display module 124 causes the display device 126 to visually present cardiac signals 402, 416 and visual indicia representative of the markers and/or heuristic information derived by the comparison module 122. In the illustrated embodiment, the visual indicia are shown at least partially overlying, or overlapping, the cardiac signals 402 in order to associate the visual indicia with different waveform segments 408 of the cardiac signals 402. In the illustrated embodiment, the cardiac signals 402 are atrial cardiac signals, or cardiac signals representative of electrical activity of one or more atria of the heart 106. The waveform segments 408 represent P-waves of the cardiac signals 402. The cardiac signals 416 are ventricular cardiac signals representative of electrical activity of one or more ventricles of the heart 106. The cardiac signals 416 include waveform segments 418 that represent R-waves in the illustrated embodiment. Alternatively, cardiac signals 402, 416 may represent electrical activity of different chambers of the heart 106 and/or different waveform segments may be shown.

The visual indicia shown in FIG. 4 are heuristic information related to the waveform segments 418 of the ventricular cardiac signals 416 and the waveform segments 408 of the atrial cardiac signals 402. With respect to the visual indicia for the ventricular cardiac signals 416, ventricular sense indicia 420 are displayed and are based on operational markers received from the IMD 102. The ventricular sense indicia 420 are displayed in positions that correspond to the detection of the waveform segments 418 by the IMD 102.

With respect to the visual indicia for the atrial cardiac signals 402, atrial sense indicia 422 are displayed and are based on operational markers received from the IMD 102. For example, the atrial sense indicia 422 may correspond to the waveform segments 408 that were detected by the IMD 102. The visual indicia also include confirmed indicia 404 and under-sensed indicia 406. The confirmed indicia 404 are shown overlying, or otherwise spatially associated with, a first subset of the waveform segments 408 of the cardiac signals 402 while the under-sensed indicia 406 are shown overlying a different second subset of the waveform segments 408. The confirmed indicia 404 indicate which waveform segments 408 are sensed by the IMD 102 and detected by the monitoring system 100. Conversely, the under-sensed indicia 406 represent the waveform segments 408 that are not sensed by the IMD 102 but are detected by the monitoring system 100.

In order to identify which waveform segments 408 are associated with the confirmed indicia 404, the waveform identification module 128 of the monitoring system 100 examines the cardiac signals 402 received from the IMD 102. The waveform identification module 128 examines the cardiac signals 402 to determine which sections of the cardiac signals 402 represent cardiac events, such as atrial or ventricular events. In one embodiment, the waveform identification module 128 may compare the cardiac signals 402 to a sensing threshold to identify waveform segments in the cardiac signals 402. The sections of the cardiac signals 402 that exceed the sensing threshold are identified as waveform segments that correspond to the threshold. For example, if the threshold used by the waveform identification module 128 is established to identify P-waves of the cardiac signals 402, then the waveform segments of the cardiac signals 402 that exceed the threshold are classified by the waveform identification module 128 as P-waves. Alternatively, a plurality of thresholds may be used and/or other waveform segments may be identified based on the thresholds.

In another embodiment, the waveform identification module 128 identifies the waveform segments 408 by comparing the cardiac signals 402 to one or more waveform templates. The waveform templates represent predetermined shapes, such as triangles, that represent one or more waveform segments of interest, such as P-waves, R-waves, QRS-complexes, and the like. In the illustrated embodiment, the waveform identification module 128 may compare the cardiac signals 402 to waveform templates representative of P-waves. The portions of the cardiac signals 402 that match the waveform templates, such as by having integrated areas, slopes, and the like, that are within a predetermined range of areas, slopes, and the like of the templates, are identified as the waveform segments 408. Alternatively, one or more other techniques may be used to identify the waveform segments 408.

The waveform identification module 128 examines the marker data received from the IMD 102 to determine if the waveform segments 408 identified by the waveform identification module 128 correspond to cardiac markers. As described above, the IMD 102 may generate a cardiac marker, such as an atrial sense cardiac marker, when the IMD 102 senses a waveform segment, such as a P-wave. The IMD 102 may sense a waveform segment when the cardiac signals exceed a sensing threshold of the IMD 102.

If there is a cardiac marker from the IMD 102 that corresponds to a waveform segment 408 identified by the waveform identification module 128, then the waveform segment 408 is associated with the confirmed indicia 404. For example, if the IMD 102 identifies a P-wave and the presence of the P-wave is confirmed by the waveform identification module 128, then the P-wave is associated with heuristic information that indicates that the P-wave is a confirmed waveform segment.

On the other hand, if the waveform identification module 128 identifies a waveform segment 408 that is not identified by the IMD 102, then the waveform identification module 128 associates the waveform segment 408 with an under-sensed indicia 406. For example, the sensing threshold of the IMD 102 may be too high such that one or more waveform segments 408 are not detected and are missed by the IMD 102 as the waveform segments 408. The under-sensed indicia 406 is associated with these missed waveform segments 408.

A physician examining the display 400 may visually identify which waveform segments 408 are missed by the IMD 102 and which waveform segments 408 are sensed by the IMD 102. Based on the number or amount of under-sensed indicia 406, the physician may adjust one or more settings of the IMD 102, such as the sensing threshold used by the IMD 102. For example, the physician may decrease the sensing threshold used by the IMD 102 to detect P-waves such that fewer P-waves are missed by the IMD 102.

The display module 124 can direct the display device 126 to present a legend 410 that provides information about the heuristic information shown in the display 400. In the illustrated embodiment, the legend 410 includes text labels 412 for the indicia 406, 408. The physician can use the text labels 412 to determine the heuristic information represented by the indicia 406, 408.

In one embodiment, after determining that the IMD 102 is potentially under-sensing cardiac signals, the comparison module 122 may examine the operational markers from the IMD 102 to determine which pacing and/or sensing algorithms are being used by the IMD 102. The memory 118 may store a look-up table or other logical database that associates heuristic information derived by the comparison module 122 with diagnostic information. For example, the heuristic information derived by the comparison module 122 may be the under-sensing of P-waves by the IMD 102, as described above. The comparison module 122 can search the look-up table or database for diagnostic information that is associated with the under-sensing of P-waves and one or more of the settings of the IMD 102. If the table or database includes diagnostic information associated with the heuristic information and algorithms, then the comparison module 122 retrieves the diagnostic information from the table or database and conveys the diagnostic information to the display module 124.

In the illustrated embodiment, the operational markers from the IMD 102 indicate the length of time of a blanking period of the IMD 102. For example, the operational markers may indicate that the IMD 102 has a post-ventricular atrial blanking (PVAB) period that lasts a predetermined time period. The PVAB period can represent a time period following detection of a ventricular event (e.g., an R-wave) that the IMD 102 does not examine the atrial cardiac signals 402 for waveform segments of interest, such as P-waves. If the PVAB period is too long and one or more P-waves occur during the PVAB period following an R-wave, then the IMD 102 may not detect or identify the P-wave. With respect to the cardiac signals 402, 416 shown in FIG. 4, the P-waves that are missed by the IMD 102 may have occurred during the PVAB period following an R-wave or a ventricular sense operational marker.

The comparison module 122 can compare the PVAB period (or other blanking period or settings of the IMD 102) to a threshold stored in the memory 118 and associated with the heuristic information derived by the comparison module 122 (e.g., the under-sensing of P-waves). For example, the identification of under-sensing of atrial waveform segments by the comparison module 122 may be associated with a PVAB period threshold in the look-up table or database in the memory 118. The comparison module 122 determines from the operational markers of the IMD 102 if the PVAB period of the IMD 102 exceeds the PVAB threshold. If the PVAB period of the IMD 102 exceeds the PVAB threshold, then the comparison module 122 retrieves diagnostic information from the table or database. For example, the comparison module 122 may retrieve and present causal traceback text 414 that is presented to the physician on the display 400. As shown in FIG. 4, the causal traceback text 414 informs the physician that the missed or under-sensed P-waves may have occurred during the PVAB period of the IMD 102. In one embodiment, the causal traceback text 414 may suggest shortening the PVAB period of the IMD 102 or provide other recommendations.

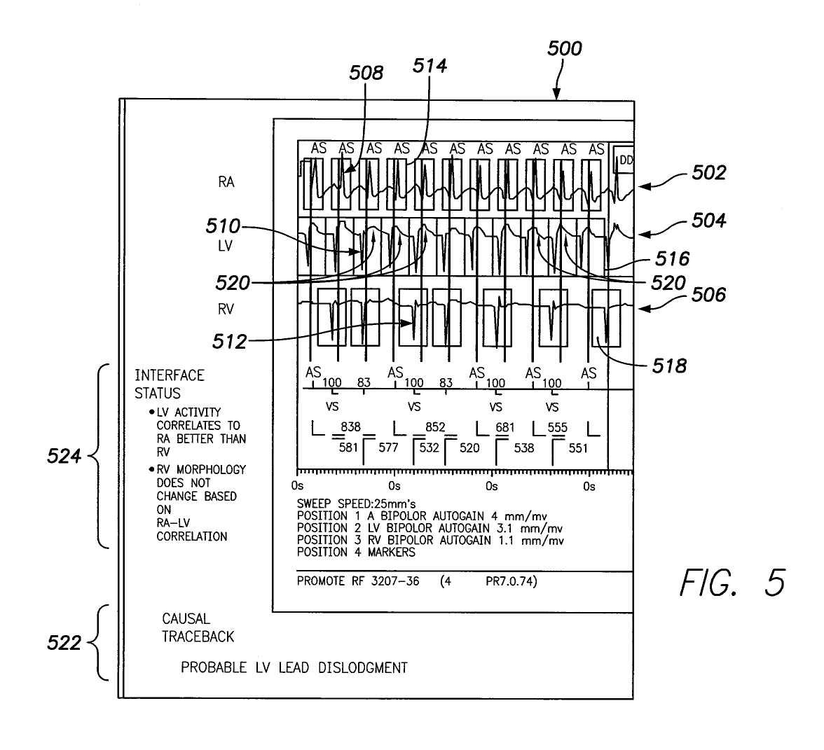

FIG. 5 is an illustration of another embodiment of a display 500 presented by the display device 126. The display 500 is another example of the visual information that may be presented by the display device 126 based on the cardiac signals and marker data from the IMD 102, as well as heuristic information (e.g., relationships between markers) derived by the comparison module 122.

In the illustrated embodiment, the display module 124 causes the display device 126 to visually present cardiac signals 502, 504, 506 and visual indicia representative of the markers and/or heuristic information derived by the comparison module 122. The visual indicia are shown at least partially overlying, or overlapping, the cardiac signals 502, 504, 506 in order to associate the visual indicia with different waveform segments 508, 510, 512 in the corresponding cardiac signals 502, 504, 506. In the illustrated embodiment, the cardiac signals 502 are cardiac signals sensed by a lead of the IMD 102 that is associated with the right atrium (e.g., was originally implanted into the right atrium) and include several P-waves as the waveform segments 508. The cardiac signals 504 are cardiac signals sensed by a lead of the IMD 102 that is associated with the left ventricle (e.g., was originally implanted in the left ventricle) and include several R-waves as the waveform segments 510. The cardiac signals 506 are cardiac signals sensed by a lead of the IMD 102 that is associated with the right ventricle (e.g., was originally implanted in the right ventricle) and include several R-waves as the waveform segments 512. Alternatively, different cardiac signals of other chambers of the heart 106 (shown in FIG. 1) and/or other waveform segments may be presented.

The visual indicia shown in FIG. 5 represent heuristic information related to the waveform segments 508, 510, 512. The visual indicia include P-wave indicia 514 and R-wave indicia 516, 518. Each of the P-wave indicia 514 is shown overlying, or otherwise spatially associated with, a different waveform segment 508 (e.g., P-wave), each of the R-wave indicia 516 overlies a different waveform segment 510 (e.g., R-wave), and each of the R-wave indicia 518 overlies a different waveform segment 512 (e.g., R-wave). The indicia 514, 516, 518 visually highlight or demark the different waveform segments 508, 510, 512 so that a physician can more easily see the waveform segments 508, 510, 512. The waveform segments 508, 510, 512 may be identified by the waveform identification module 128, as described above, and the comparison module 122 may direct the display module 124 to display the corresponding indicia 514, 516, 518 on the waveform segments 508, 510, 512. Alternatively, the waveform segments 508, 510, 512 may be identified by the comparison module 122 examining the cardiac markers generated by the IMD 102. For example, the comparison module 122 may direct the display module 124 to display the indicia 514, 516, 518 each time the cardiac marker of a corresponding waveform segment 508, 510, 512 is generated by the IMD 102. The visual indicia 514, 516, 518 can assist a physician to more clearly see waveform segments of interest in the cardiac signals 502, 504, 506.

In one embodiment, the comparison module 122 examines one or more of the cardiac signals 502, 504, 506 and/or the marker data associated with the cardiac signals 502, 504, 506 to identify correlations between the waveform segments 508, 510, 512. The comparison module 122 may compare the frequency and/or times at which waveform segments associated with different chambers of the heart 106 occur to determine if a correlation exists between the different chambers. For example, with respect to the illustrated embodiment, the comparison module 122 may determine if the frequency and/or times at which the left ventricular waveform segments 510 (e.g., R-waves) occur correspond with the frequency and/or times at which the right ventricular waveform segments 512 (e.g., R-wave) occur. As shown in FIG. 5, the right ventricular waveform segments 512 occur at a smaller frequency and at different times than the left ventricular waveform segments 510. As a result, the comparison module 122 determines that the right ventricular waveform segments 512 are not correlated with the left ventricular waveform segments 510. This determination may be heuristic information that is derived by the comparison module 122 and presented on the display 500.

The waveform identification module 128 can analyze the cardiac signals 502, 504, 506 to identify additional waveform segments that are not detected by the IMD 102. For example, the left ventricular cardiac signals 504 illustrate trailing waveform segments 520 that follow the waveform segments 510 (e.g., R-waves). The waveform identification module 128 can identify the trailing waveform segments 520 using one or more of a variety of techniques. As described above, the waveform identification module 128 can identify one or more of the trailing waveform segments 520 when the cardiac signals 504 exceed one or more thresholds, when the shape and/or area of a section of the cardiac signals 504 matches one or more predetermined waveform templates, and the like. The identification and/or location of the trailing waveform segments 520 may be heuristic information that is derived by the waveform identification module 128.

The comparison module 122 can use the heuristic information (e.g., the timing and/or location of the trailing waveform segments 520) to determine diagnostic information relevant to the IMD 102. For example, the comparison module 122 can compare the timing and/or location of the waveform segments 510, 520 in the left ventricular cardiac signals 504 with the timing and/or location of the waveform segments 508, 512 in the right atrium cardiac signals 502 and the right ventricular cardiac signals 506, respectively. As shown in FIG. 5, the trailing waveform segments 520 in the left ventricular cardiac signals 504 correlate more with the waveform segments 508 (e.g., P-waves) of the right atrium cardiac signals 502 than the waveform segments 512 in the right ventricular cardiac signals 506. For example, the timing and/or location of the trailing waveform segments 520 may match the timing and/or location of the waveform segments 520 than the waveform segments 512. Moreover, the comparison module 122 may determine that the waveform segments 512 (e.g., R-waves) of the right ventricular cardiac signals 506 do not correlate, or have relatively low correlation, with the waveform segments 508, 510, 520 in the right atrium and left ventricular cardiac signals 502, 504. The correlation and/or lack of correlation between waveform segments in different cardiac signals is heuristic information that is derived by the comparison module 122. The correlation and/or lack of correlation may be expressed as a degree of match between the waveform segments in the cardiac signals, as described above.

The comparison module 122 can refer to a diagnostic knowledge base or knowledge domain stored in the memory 118. The knowledge base may be embodied in sets of heuristic rules, criteria, thresholds, and the like, which are associated with diagnoses. The heuristic information is compared to the heuristic rules or criteria to determine if the heuristic information satisfies the rules or criteria. If the heuristic information satisfies a heuristic rule or criteria, then the comparison module 122 may retrieve the diagnosis associated with the heuristic rule or criteria so that the diagnosis can be presented to the physician.

By way of example, a heuristic rule may specify that if cardiac signals from a first ventricle do not correlate with the cardiac signals from a second ventricle but do correlate with the cardiac signals from an atrium, then a potential cause for the correlation and lack of correlation may be a dislodged ventricular lead. The heuristic rule may specify thresholds to which the degrees of match between cardiac signals are compared. The cardiac signals having degrees of match that exceed the threshold may be correlated with each other while the cardiac signals that have degrees of match that do not exceed the threshold may not be correlated with each other. In the illustrated embodiment, the comparison module 122 may determine that the left ventricular cardiac signals 504 satisfy the heuristic rule because the left ventricular cardiac signals 504 are correlated with the right atrium cardiac signals 502 but not with the right ventricular cardiac signals 506. As a result, the comparison module 122 retrieves diagnostic information associated with the heuristic rule.

The diagnostic information can be presented to the physician using the monitoring system 100. In the illustrated embodiment, the diagnostic information includes causal traceback text 522 and heuristic rule summary text 524. The heuristic rule summary text 524 describes to the physician that the left ventricular cardiac signals 504 are more correlated with the right atrium cardiac signals 502 than the right ventricular cardiac signals 506 and that the morphology or shape of the right ventricular cardiac signals 506 appears to be independent of the correlation between the right atrium cardiac signals 502 and the left ventricular cardiac signals 504. The causal traceback text 522 describes to the physician that the potential cause for the cardiac signals 502, 504, 506 may be a dislodged left ventricular lead of the IMD 102. For example, a lead of the IMD 102 may have become decoupled from the left ventricle of the heart 106 and may have moved into the right atrium such that the lead is sensing right atrium cardiac signals.

The displays 300, 400, 500 (FIGS. 3 through 5) shown and described above provide examples of one or more embodiments of the disclosed subject matter. In general, heuristic information such as conclusions and hypotheses is derived from literal data (e.g., cardiac signals and marker data) obtained from the IMD 102. The heuristic information can be visually presented to a physician as visual indicia to more clearly identify features (e.g., waveform segments, delivery of stimulus pulses) in the literal data. Different visual indicia may be turned off or on by use of the user interface 130. For example, a physician may select which heuristic information is represented by visual indicia and which heuristic information is not shown on the display 300, 400, 500.

The cardiac signals, heuristic information, and/or visual indicia representative of the heuristic information may be presented in real time, or during the same time period that the cardiac signals are obtained from the patient 108. For example, the cardiac signals, heuristic information, and/or visual indicia may be presented on the display device 126 as the cardiac signals are concurrently obtained from the patient 108. As the cardiac signals scroll across the display device 126, the visual indicia may be presented on the display device 126. Alternatively, the cardiac signals, heuristic information, and/or visual indicia may be displayed after the cardiac signals are obtained. For example, the cardiac signals may be acquired from the IMD 102 and later analyzed and presented on the display device 126.

The description of heuristic information and diagnostic information that is derived and provided based on the literal data acquired from a medical device is provided as a few examples. Other heuristic information and diagnostic information may be derived and provided. The heuristic information and diagnostic information may be updated or changed by users of the monitoring system 100. For example, the user interface 130 of the monitoring system 100 may be used by a physician to provide new or updated diagnostic information based on information learned by the physician through empirical studies, clinical studies, experience, and the like. Alternatively, the diagnostic information may be remotely updated over or through a network connection with the monitoring system 100. For example, the monitoring system 100 may periodically download diagnostic information from a server or other computer over the Internet.

In addition to or as an alternate to the heuristic information and diagnostic information described above, the monitoring system 100 may examine the literal data obtained from the IMD 102 to identify marker patterns that are sub-optimal. The identification of certain marker patterns as sub-optimal is another example of heuristic information. By “sub-optimal,” it is meant that the marker pattern is not correlated with (e.g., has a degree of match below a threshold) one or more predetermined patterns stored in the memory 118 and that are associated with hemodynamic performance or cardiac output above an associated threshold or that at least meets industry or medical standards. The predetermined patterns may be created and/or updated by physicians.

In another example, the monitoring system 100 may examine the literal data to identify inconsistent waveform morphology in cardiac signals. For example, the monitoring system 100 can analyze the shape of waveform segments in cardiac signals to determine if the shape of the waveform segments change or vary over time. Changing or varying waveform segments may be indicative of cardiac disease, sub-optimal marker patterns, sub-optimal settings of the IMD 102, and the like. With respect to the settings of the IMD 102, “sub-optimal” means that the settings of the IMD 102 may cause the hemodynamic performance or cardiac output of the heart 106 to be above an associated threshold or at least meets industry or medical standards.

In another example of heuristic information, the monitoring system 100 may examine the literal data to identify a loss of capture of a stimulus pulse. For example, the monitoring system 100 may determine a loss of capture when a cardiac event does not follow delivery of a stimulus pulse within a predetermined time period. The stimulus pulse can be identified by the operational markers from the IMD 102 and the lack of a cardiac event may be identified by examination of the cardiac signals and cardiac markers.

The monitoring system 100 provides recommended changes to the settings and/or algorithms of the IMD 102 based on the derived heuristic information and/or diagnostic information in one embodiment. For example, if the derived heuristic information indicates that a sensing threshold of the IMD 102 is set too high, the monitoring system 100 may display a recommended reduction in the sensing threshold on the display device 126. Alternatively, if the heuristic information indicates that a blanking period is too long, the monitoring system 100 may present a shorter blanking period. Additional recommendations may be provided based on the heuristic or diagnostic information, such as recommended algorithms, other changes to the settings, and the like.

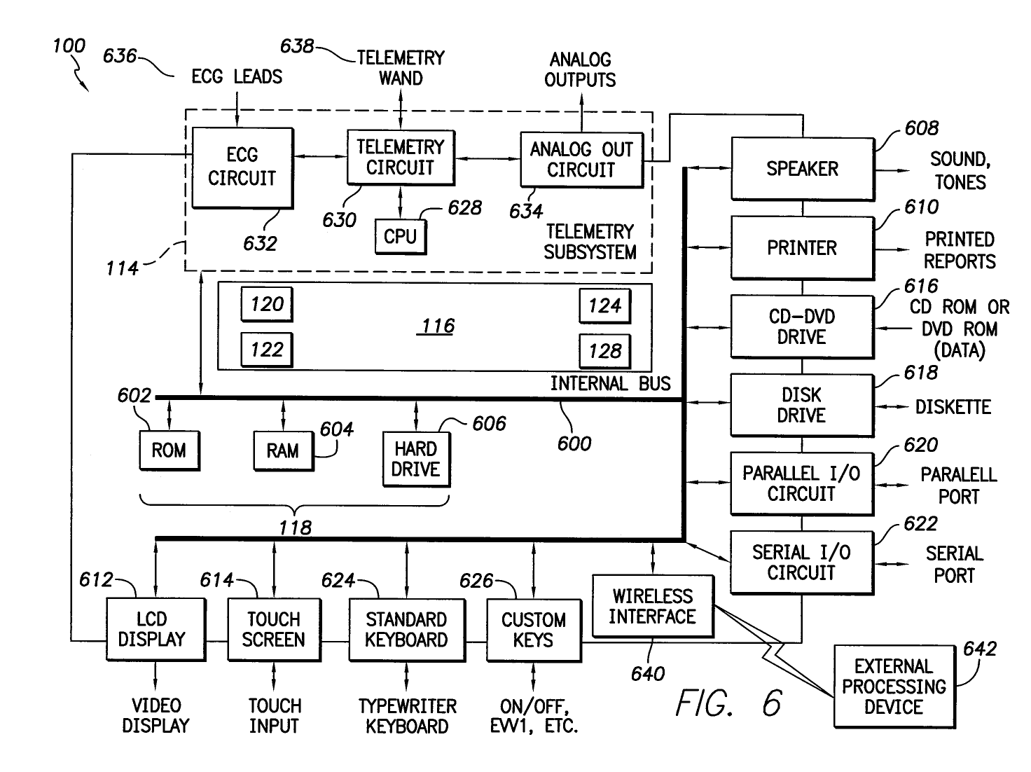

FIG. 6 illustrates a functional block diagram of one embodiment of the monitoring system 100. The monitoring system 100 includes an internal bus 600 that connects/interfaces with the processor 116 and the memory 118. In the illustrated embodiment, the memory 118 includes ROM 602, RAM 604, and a hard drive 606. Alternatively, the memory 118 may include a different combination of tangible and non-transitory computer readable storage media or different media. As described above, the processor 116 includes the modules 120, 122, 124, 128 that operated based on instructions stored on the memory 118.

The monitoring system 100 may include output devices such as a speaker 608, a printer 610, and an LCD display 612 joined to the internal bus 600. One or more input/output devices also may be included. Such devices include a touch screen 614, a CD/DVD drive 616, a disk drive 618, a parallel I/O circuit 620, and a serial I/O circuit 622. The display device 126 may be embodied in one or more of the LCD display 612 and/or the touch screen 614. The internal bus 600 is an address/data bus that transfers information between the various components described herein.

The touch screen 614 accepts touch input from a physician when selections are made. For example, a physician may touch visual indicia corresponding to heuristic information displayed on the touch screen 614 to toggle between displaying or not displaying the visual indicia. A keyboard 624 (e.g., a typewriter keyboard) allows the physician to enter data to the displayed fields, as well as interface with the communication subsystem 114. For example, a physician can input diagnostic information that may be associated with heuristic information derived from literal data that has not yet been acquired. Furthermore, custom keys 626 can be used to turn the monitoring system 100 on or off.

The printer 610 can print copies of reports for a physician to review or to be placed in a patient file. The speaker 608 provides an audible warning (e.g., sounds and tones) to the physician. The parallel I/O circuit 620 interfaces with a parallel port to transfer data therebetween. The serial I/O circuit 622 interfaces with a serial port to transfer data therebetween. The disk drive 618 accepts disks or USB devices. The CD/DVD drive 616 accepts CDs and/or DVDs. The user interface 130 and/or input device 132 shown in FIG. 1 may include or be embodied in one or more of the input and input/output devices, such as the touch screen 614, keyboard 624, or custom keys 626.

In the illustrated embodiment, the communication subsystem 114 includes a central processing unit (CPU) 628 in electrical communication with a telemetry circuit 630, which communicates with both an ECG circuit 632 and an analog out circuit 634. The ECG circuit 632 can be connected with ECG leads 636, such as leads coupled with an ECG medical device. The telemetry circuit 630 is connected to a telemetry wand 638. The analog out circuit 634 includes communication circuits to communicate with analog outputs. The communication subsystem 114 may wirelessly communicate with the IMD 102 and utilize protocols, such as Bluetooth, GSM, infrared wireless LANs, HIPERLAN, 3G, satellite, as well as circuit and packet data protocols, and the like. Alternatively, a hard-wired connection may be used to connect the monitoring system 100 to the IMD 102.

A wireless interface 640, such as a transceiver unit, is coupled to the bus 600 and communicatively coupled with the processor 116. The wireless interface 640 communicates data, such as cardiac signals, marker data, and the like, with an external processing device 642. The processing device 642 may be a computing device that can receive and analyze the cardiac signals and marker data to display the data, derive and/or process the heuristic information, and/or display the heuristic information to a physician, as described above. Alternatively, the analysis and/or processing of data to derive the heuristic information can be shared by the processing device 642 and the system 100. The processing device 642 can wirelessly transmit the heuristic information to the monitoring system 100 via the wireless interface 640. By way of example only, the processing device 642 may include one or more tablet computers, smart phones, personal digital assistants (PDAs) and the like.

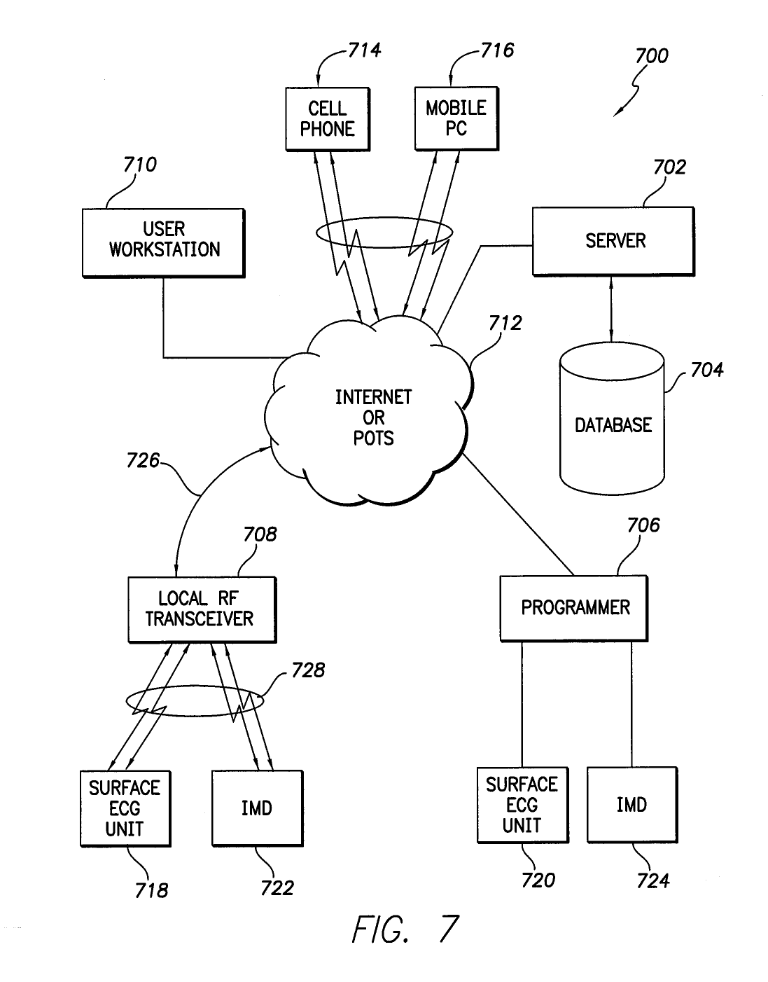

FIG. 7 illustrates a distributed processing system 700 in accordance with one embodiment. The distributed processing system 700 includes a server 702 connected to a database 704, a programmer 706 (for example, similar to the monitoring system 100 shown in FIG. 1), a local RF transceiver 708, and a user workstation 710 electrically connected to a communication system 712.

The communication system 712 may be the Internet (or a portion thereof), an intranet, a voice over IP (VoIP) gateway, a local plain old telephone service (POTS) such as a public switched telephone network (PSTN), a cellular phone based network, and the like. Alternatively, the communication system 712 may be a local area network (LAN), a campus area network (CAN), a metropolitan area network (MAN), or a wide area network (WAM). The communication system 712 serves to provide a network that facilitates the transfer/receipt of literal data, heuristic information, and/or diagnostic information, among other data and information.

The server 702 is a computer system that provides services to other computing systems over a computer network. The server 702 may control the communication of information such as literal data, heuristic information, heuristic rules, and/or diagnostic information. The server 702 interfaces with the communication system 712 to transfer information between the programmer 706, the local RF transceiver 708, the user workstation 710, as well as a cell phone 714, and a mobile computing device 716 (“mobile PC”) to the database 704 for storage/retrieval of literal data, heuristic information, heuristic rules, and/or diagnostic information. On the other hand, the server 702 may upload literal data from a surface ECG unit 718, 720 or a medical device 722, 724 (MD), such as the IMD 102 shown in FIG. 1, via the local RF transceiver 708 or the programmer 706. The mobile computing device 716 may include one or more of a PDA, tablet computer, smart phone, and the like. In one embodiment, the server 702 may process the cardiac signals and marker data to derive heuristic information for an operator of the system 100. For example, the server 702 may be remote from a physician (e.g., located in a different floor, building, city, county, state, or country) and receive cardiac signals and marker data via the communication system 712. The server 702 may process the cardiac signals and the marker data as described above, such as by comparing the cardiac signals and the marker data to one or more heuristic rules to determine which, if any, rules are satisfied by the cardiac signals and marker data in order to derive heuristic information about the cardiac signals and marker data. The server 702 may then communicate the heuristic information to the physician using the user workstation 710, for example.

The database 704 may store literal data, heuristic information, heuristic rules, and/or diagnostic information for a single or multiple patients. The literal data, heuristic information, heuristic rules, and/or diagnostic information can be downloaded into the database 704 via the server 702 or, alternatively, the information is uploaded to the server 702 from the database 704. The programmer 706 may reside in a patient's home, a hospital, or a physician's office. The programmer 706 can interface with the surface ECG unit 720 and/or the medical device 724. The programmer 706 may wirelessly communicate with the medical device 724 and utilize protocols, such as Bluetooth, GSM, infrared wireless LANs, HIPERLAN, 3G, satellite, as well as circuit and packet data protocols, and the like. Alternatively, a hard-wired connection may be used to connect the programmer 706 to the medical device 724. The programmer 706 is able to acquire cardiac signals from the surface of a person (e.g., ECGs), intra-cardiac electrogram (e.g., IEGM) signals, and/or marker data from the medical device 724. The programmer 706 interfaces with the communication system 712, either via the Internet or via POTS, to upload the information acquired from the surface ECG unit 718, 720 or the medical device 722, 724 to the server 702.

The local RF transceiver 708 interfaces with the communication system 712 via a communication link 726, to upload literal data acquired from the surface ECG unit 718 and/or the medical device 722 to the server 702. In one embodiment, the surface ECG unit 718 and the medical device 722 have a bi-directional connection with the local RF transceiver 708 via a wireless connection 728. The local RF transceiver 708 is able to acquire cardiac signals from the surface of a person, intra-cardiac electrogram signals from the medical device 722, and/or marker data from the medical device 722.

The user workstation 710 may interface with the communication system 712 via the Internet or POTS to download literal data, heuristic information, and/or diagnostic information via the server 702 from the database 704. Alternatively, the user workstation 710 may download literal data from the surface ECG unit 718, 720 or medical device 722, 724 via either the programmer 706 or the local RF transceiver 708. Once the user workstation 710 has downloaded the literal data, heuristic information, and/or diagnostic information, the user workstation 710 may process the literal data in accordance with one or more of the operations described above to derive heuristic information and/or diagnostic information. Alternatively, the user workstation 710 may download the literal data from a medical device or ECG and derive the heuristic information and determine associated diagnostic information from the database 704. The user workstation 710 may download the information and supply the literal data, heuristic information, and/or diagnostic information to the cell phone 714, the PDA 716, the local RF transceiver 708, the programmer 706, or to the server 702 to be stored on the database 704.

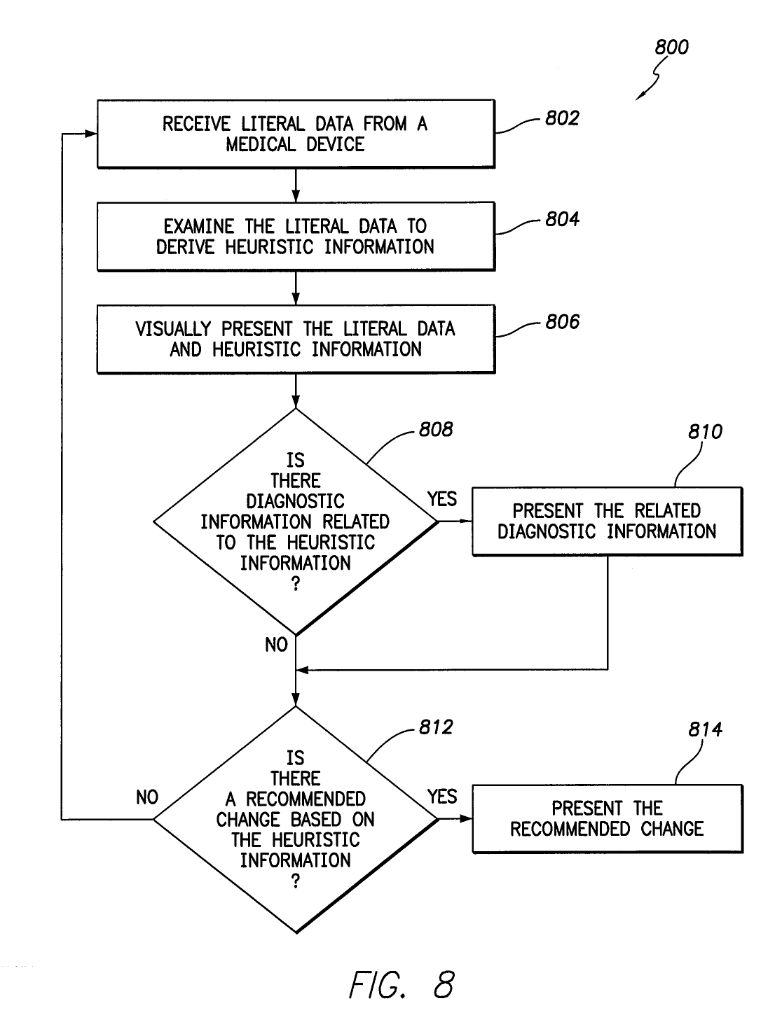

FIG. 8 is a flowchart of one embodiment of a method 800 for monitoring a medical device. The method 800 may be used to analyze literal data (e.g., cardiac signals and marker data) acquired or generated by a medical device, such as an ECG/EKG device, implantable medical device, and the like. The method 800 can be used to derive heuristic information and associated diagnostic information from the literal data, as described above. The heuristic and diagnostic information can assist a physician in identifying poor hemodynamic performance of the heart and/or medical device settings or algorithms that can be changed to improve the hemodynamic performance.

At 802, literal data is received from a medical device. For example, the IMD 102 may sense cardiac signals of the heart 106 and generate operational markers based on the cardiac signals and/or operations of the IMD 102. The literal data may be analyzed in accordance with the method 800 in real time (e.g., as the literal data is acquired) or at a later time (e.g., stored for a relatively extended period of time before analysis).

At 804, the literal data is examined to determine heuristic information. As described above, the heuristic information may include characteristics of or conclusions made based on the literal data. By way of example only, heuristic information can include identification of waveform segments, marker patterns, under-sensed waveform segments, sub-optimal marker patterns, sub-optimal settings of the medical device, algorithms of the medical device that interfere with each other, and the like.

At 806, the literal data and the heuristic information are visually presented. As described above, the literal data and the heuristic information may be displayed on the display device 126. The heuristic information can be presented as visual indicia, such as highlighted shapes, that overlie the cardiac signals of the literal data. The heuristic information can assist physicians in identifying patterns, waveform segments, and other characteristics of the literal data. The literal data and heuristic information can be presented in real time, such as during at least part of the same time period that the literal data is acquired from the patient 108. Alternatively, the literal data and/or heuristic information may be acquired and stored for presentation after the literal data is acquired from the patient 108.

At 808, a determination is made as to whether there is diagnostic information that is related to the heuristic information. As described above, some heuristic information may be indicative of a diagnosis, such as an identification of a cardiac event, sub-optimal settings of the medical device, interfering algorithms of the medical device, and the like. The heuristic information can be compared to one or more criteria or heuristic rules associated with the diagnostic information. If the heuristic information satisfies or meets the criteria or heuristic rules, then the associated diagnostic information is related to the heuristic information, as described above. As a result, flow of the method 800 proceeds to 810. Conversely, if no diagnostic information is related to the heuristic information, then flow of the method 800 proceeds to 812.

At 810, the diagnostic information is presented. For example, the diagnostic information can be displayed on the display device 126 as causal traceback text or other text. Alternatively, the diagnostic information may be presented in a graphical and/or non-textual manner.

At 812, a determination is made as to whether there is a recommended change based on the heuristic information. As described above, some heuristic information may be indicative of a setting, threshold, or algorithm of the IMD 102 that may need to be changed to alter the hemodynamic performance of the heart 106. The recommended change may be part of the diagnostic information that is associated with certain heuristic information. For example, the heuristic information can be compared to one or more criteria or heuristic rules associated with the recommended change. If the heuristic information satisfies or meets the criteria or heuristic rules, then the heuristic information indicates that the recommended change is needed to fix or correct the IMD 102 and/or hemodynamic performance of the heart 106. As a result, flow of the method 800 proceeds to 814. Conversely, if the heuristic information does not indicate that a change is needed, then flow of the method 800 returns to 802, where additional literal data may be received.

At 814, the recommended change is presented. For example, the recommended change may be visually presented on the display device 126 as text or other instructions to a physician. The recommended change can be presented along with visual indicia of which settings, thresholds, or algorithms of the IMD 102 are to be changed or adjusted by the recommended change. The recommended change also may instruct the physician how to adjust the settings, thresholds, or algorithms.

As used throughout the specification and claims, the phrases “computer-readable medium” and “instructions configured to” shall refer to any one or all of (i) computer-readable media or memory, software source code, software object code, hard wired logic, and/or software applications that direct processors, microprocessors, microcontrollers, and the like, to perform one or more directed operations.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the disclosed subject matter without departing from its scope. While the dimensions and types of materials described herein are intended to define the parameters of the described subject matter, they are by no means limiting and are exemplary embodiments. Many other embodiments will be apparent to one of ordinary skill in the art upon reviewing the above description. The scope of the claimed subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person of ordinary skill in the art to practice the described subject matter, including making and using any devices or systems and performing any incorporated methods. The patentable scope of the disclosed subject matter is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

1. A cardiac monitoring system comprising:
a cardiac medical device configured to be associated with a patient's heart and to generate literal data including cardiac signals and marker data, the cardiac signals representative of electrical activity of the heart as sensed by the cardiac medical device;
a communication subsystem configured to receive literal data from the cardiac medical device;
a comparison module configured to compare one or more characteristics of the literal data to one or more heuristic rules in order to derive heuristic information about the literal data, wherein the heuristic information includes 1) a diagnosis relating to at least one of a health status of the patient and the operation of the cardiac medical device, and 2) a causal traceback corresponding to the cause of the diagnosis; and
a display module configured to direct a display device to visually present the cardiac signals and visual indicators representative of the heuristic information.

2. The cardiac monitoring system of claim 1, further comprising a memory storing a knowledge base comprised of a plurality of the heuristic rules and their corresponding heuristic information, wherein the comparison module is configured to compare the one or more characteristics of the literal data with one or more of the heuristic rules and retrieve the heuristic information corresponding to the one or more heuristic rules when the one or more characteristics of the literal data satisfies the one or more of the heuristic rules.

3. The cardiac monitoring system of claim 2, wherein the heuristic information relates to the operation of the cardiac medical device and includes a recommended change to one or more of a setting of the medical device, a sensing algorithm of the medical device, or a pacing algorithm of the medical device.

4. The cardiac monitoring system of claim 2, wherein the heuristic information relates to a diagnosis and represents a potential medical cause of the literal data.

5. (canceled)

6. The cardiac monitoring system of claim 2, wherein the heuristic information relates to the operation of the cardiac medical device and is indicative of at least one of under-sensing or over-sensing of the cardiac events by the medical device.

7. (canceled)

8. The cardiac monitoring system of claim 1, further comprising a sequence detection module configured to examine the marker data and identify a marker pattern over a period of time, wherein the marker pattern corresponds to the one or more characteristics of the literal data.

9. The cardiac monitoring system of claim 1, wherein one or more of the heuristic rules are modifiable by an operator of the system.

10. A method for monitoring a cardiac medical device associated with a patient's heart, the method comprising:
receiving literal data from the medical device, the literal data including cardiac signals and marker data, the cardiac signals representative of electrical activity of a heart sensed by the medical device;
comparing one or more characteristics of the literal data to one or more heuristic rules to derive heuristic information about the literal data, wherein the heuristic information includes 1) a diagnosis relating to at least one of a health status of the patient and the operation of the cardiac medical device, and 2) a causal traceback corresponding to the cause of the diagnosis; and
visually presenting the cardiac signals and a visual indicator representative of the heuristic information.

11. The method of claim 10, further comprising storing a knowledge base comprised of a plurality of the heuristic rules and their corresponding heuristic information, wherein deriving the heuristic information includes comparing the the one or more characteristics of the literal data with one or more of the heuristic rules, and retrieving the heuristic information corresponding to the one or more heuristic rules when the one or more characteristics of the literal data satisfies the one or more of the heuristic rules.

12. The method of claim 11, wherein the heuristic information relates to the operation of the cardiac medical device and includes a recommended change to one or more of a setting of the medical device, a sensing algorithm of the medical device, or a pacing algorithm of the medical device.

13. The method of claim 11, wherein the heuristic information relates to a diagnosis and represents a potential medical cause of the literal data from which the heuristic information is derived.

14. (canceled)

15. The method of claim 11, wherein the heuristic information relates to the operation of the cardiac medical device and is indicative of at least one of under-sensing or over-sensing of the cardiac events by the medical device.

16. The method of claim 10, wherein visually presenting the cardiac signals and the visual indicator includes displaying the visual indicator as a colored or shaded portion of a display that at least partially overlie the cardiac signals.

17. The method of claim 10, further comprising examining the marker data and identifying a marker pattern over a period of time, wherein the marker pattern corresponds to the one or more characteristics of the literal data.

18. The method of claim 10, wherein visually presenting the cardiac signals and the visual indicator includes displaying the cardiac signals and visual indicator while the cardiac signals are sensed by the medical device.

19.-25. (canceled)
